(19) United States
(12) Patent Application Publication
(10) Pub. No.: US 2016/0097132 A1
(43) Pub. Date: Apr. 7, 2016

(54) REUSABLE SPRAY BOTTLE WITH INTEGRATED DISPENSER

(71) Applicant: Microlin, LLC, Salt Lake City, UT (US)

(72) Inventors: Ashok V Joshi, Salt Lake City, UT (US); Sai Bhavaraju, West Jordan, UT (US)

(73) Assignee: Microlin, LLC, Salt Lake City, UT (US)

(21) Appl. No.: 14/874,042

(22) Filed: Oct. 2, 2015

Related U.S. Application Data
(60) Provisional application No. 62/059,297, filed on Oct. 3, 2014.

(51) Int. Cl. C25B 1/24; C25B 9/00; B01J 19/12

(52) U.S. Cl. CPC C25B 1/24; B01J 19/123; C25B 9/00; B01J 2219/0801; B01J 2219/0877; B01J 2219/1203

(57) ABSTRACT

An apparatus for cleaning and/or disinfecting surfaces and objects includes a spray bottle that is refillable with an aqueous solution, the spray bottle including a nozzle and a container. The apparatus further includes a conduit in communication with the nozzle and an interior of the container, an actuator for pumping the aqueous solution from the interior of the container to outside the spray bottle through the nozzle, an ultraviolet light source in communication with the conduit configured to at least partially radiate the aqueous solution in the conduit, a power source in communication with the ultraviolet light source, and an actuator in electrical communication with the power source, the actuator configured to provide power to the ultraviolet light source upon actuation of the actuator.

### RELATED APPLICATIONS

This application claims benefit of U.S. Provisional Application No. 62/059,297, filed Oct. 3, 2014, which is incorporated herein by reference.

### FIELD OF THE INVENTION

This invention relates to apparatus and methods for cleaning and/or disinfecting surfaces and objects.

### BACKGROUND

The global market for cleaning and disinfecting products and equipment is large and growing, on the order of tens of billions of dollars every year. For example, the global market for industrial and institutional cleaning products is forecast to exceed $36.7 billion by the year 2015. The U.S. represents the largest regional market for industrial and institutional cleaning products, with Europe coming in second. Increased safety and health standards in the food and beverage, food service, and health care sectors, where hygienic environments are required, are driving growth for industrial and institutional cleaning products and equipment.

Currently, a wide range of products and equipment are available to clean and disinfect surfaces and objects in residential, industrial, commercial, hospital, hotel, food processing, and restaurant environments. Unfortunately, some of the best products and equipment for cleaning and disinfecting are confined to the commercial or industrial marketplaces due to their increased expense. That is, the small household user typically cannot afford or justify the expense associated with purchasing and maintaining commercial-quality cleaning products and equipment. Thus, although a substantial need exists for cleaning and disinfecting surfaces and objects in residential settings, typical household users may not have the best and most effective products and equipment at their disposal.

In view of the foregoing, what are needed are products and equipment for cleaning and/or disinfecting surfaces and objects in residential and other similar settings. Ideally, such products and equipment will provide results comparable to products and equipment used in industrial and/or commercial settings but without the associated costs. Further needed are products and equipment that are reusable many times without having to replenish the active agents used for cleaning and/or disinfecting. Yet further needed are water-based cleaners as opposed to solvent-based cleaners. Such water-based cleaners may reduce the environmental, safety, and health concerns associated with solvent-based cleaners.

### SUMMARY

The invention has been developed in response to the present state of the art and, in particular, in response to the problems and needs in the art that have not yet been fully solved by currently available apparatus and methods. Accordingly, the invention has been developed to provide apparatus and methods to clean and/or disinfect surfaces and objects. The features and advantages of the invention will become more fully apparent from the following description and appended claims, or may be learned by practice of the invention as set forth hereinafter.

Consistent with the foregoing, an apparatus for cleaning and/or disinfecting surfaces and objects is disclosed herein. In one embodiment, an apparatus for cleaning and/or disinfecting surfaces and objects includes a spray bottle that is refillable with an aqueous solution, the spray bottle including a nozzle and a container. The apparatus further includes a conduit in communication with the nozzle and an interior of the container, an actuator for pumping the aqueous solution from the interior of the container to outside the spray bottle through the nozzle, an ultraviolet light source in communication with the conduit configured to at least partially radiate the aqueous solution in the conduit, a power source in communication with the ultraviolet light source, and an actuator in electrical communication with the power source, the actuator configured to provide power to the ultraviolet light source upon actuation of the actuator.

In another embodiment, an apparatus for cleaning and/or disinfecting surfaces and objects includes a spray bottle that is refillable with water, wherein the spray bottle includes an ultra violet light source, and a dispenser integrated into the spray bottle to dispense a soluble material into the water to produce a solution, the soluble material including one of a cleaning agent and a disinfecting agent. The soluble material is provided in a quantity sufficient to last several refills of the spray bottle. The ultra violet light source is configured to at least partially radiate the solution to create a plurality of hydroxyl radicals in the solution. Other embodiments of an apparatus for cleaning and/or disinfecting surfaces and objects are also described.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 shows an embodiment of a spray bottle with an integrated dispenser, wherein the integrated dispenser is configured to dispense pellets or tablets into the spray bottle;

FIG. 2 shows an embodiment of a battery-powered electrolyzer incorporated into a spray bottle with an integrated dispenser;

FIG. 3 shows an embodiment of a generator-powered electrolyzer incorporated into a spray bottle with an integrated dispenser;

FIG. 4 shows an embodiment of a spray bottle with an integrated dispenser, wherein the integrated dispenser is configured to dispense a liquid or gel into the spray bottle;

FIGS. 5A and 5B show several examples of electrolyzers and exemplary input and output streams;

FIG. 6 shows one embodiment of an electrochemical cell that may be used to produce “activated” water containing hydrogen peroxide; and

FIG. 7 shows an embodiment of a battery-powered ultraviolet light source incorporated into a spray bottle.

FIG. 8 shows an embodiment of an apparatus for cleaning and/or disinfecting surfaces and objects.

FIG. 9 shows an embodiment of an apparatus for cleaning and/or disinfecting surfaces and objects.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

In the following description, specific details of various embodiments are provided. However, some embodiments may be practiced without at least some of these specific details. In other instances, certain methods, procedures, components, and circuits are not described in detail for the sake of brevity and clarity.

The present invention may be embodied in other specific forms without departing from its spirit or essential characteristics. The described embodiments are to be considered in all respects only as illustrative and not restrictive. While the various aspects of the embodiments are presented in drawings, the drawings are not necessarily drawn to scale unless specifically indicated. The scope of the invention is, therefore, indicated by the appended claims rather than by the foregoing description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

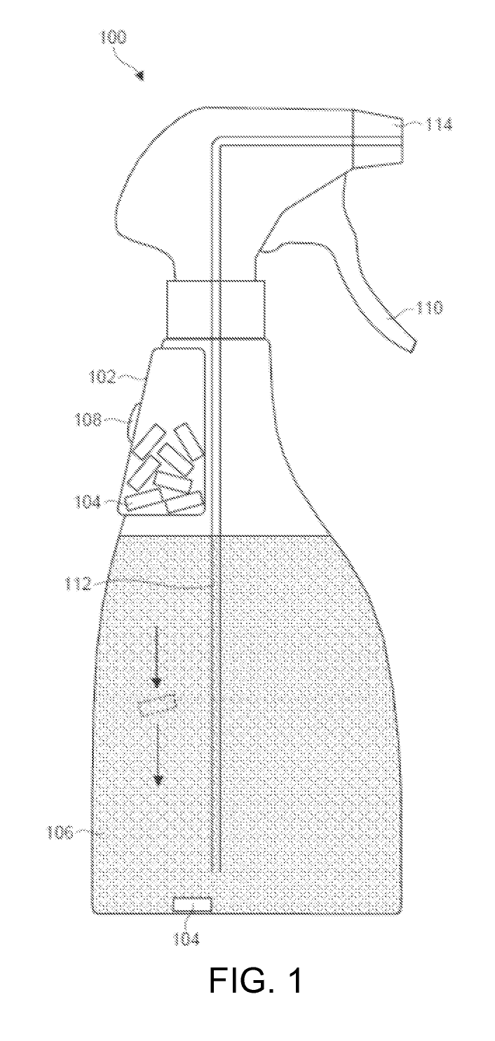

Referring to FIG. 1, one embodiment of a spray bottle 100 with integrated dispenser 102 is illustrated. In this embodiment, the integrated dispenser 102 is configured to dispense pellets 104 or tablets 104 into the spray bottle 100. These pellets 104 or tablets 104 may include a cleaning agent and/or disinfecting agent that dissolves in or mixes with water to form a solution 106. The solution 106 may have disinfecting/cleaning properties or have such properties after the solution is passed through an electrolyzer or an electrochemical cell. In one embodiment, the mixture of water and pellets forms a solution 106 and that solution 106 is electrolyzed according to the following formula:

2O₃+2H₂O₂+2H₂O→4OH.+O₂+O₃+H₂O₂+H₂O

Where OH. is a hydroxyl radical. Embodiments of the spray bottle 100 comprising an electrolyzer and/or electrochemical cell will be discussed in more detail hereafter.

As shown, in certain embodiments, the integrated dispenser 102 may be configured to release pellets 104 or tablets 104 into the spray bottle 100 so that the pellets 104 or tablets 104 can dissolve in or mix with water. In certain embodiments, the integrated dispenser 102 includes a button 108 or other actuator to enable a user to release one or more pellets 104 or tablets 104 into the water. One benefit of this arrangement is that, when the solution 106 has been depleted, the user does not have to refill the spray bottle 100 with cleaning and/or disinfecting solution but rather only water. The pellets 104 or tablets 104 will be effective to convert the water into a cleaning and/or disinfecting solution 106. Furthermore, the integrated dispenser 102 may contain enough pellets 104 or tablets 104 for multiple refills of the spray bottle 100. Thus, the user will only need to have water at his or her disposal to refresh the spray bottle 100 with cleaning and/or disinfecting solution 106.

The pellets 104 or tablets 104 may contain various chemicals to provide desired disinfecting and/or cleaning properties. For example, in certain embodiments, the pellets 104 or tablets 104 contain one or more of soluble chlorites (e.g., metal chlorites), soluble hypochlorites (e.g., metal hypochlorites), soluble halides (e.g., metal halides), ammonium salts, or the like. Each of these compounds, when dissolved in or mixed with water, may produce solutions 106 having cleaning and/or disinfecting properties. For example, sodium hypochlorite, when dissolved in water, produces bleach, commonly used as a disinfectant or bleaching agent. Ammonium salts (e.g., ammonium carbonate) may dissolve in water to form a solution and, after passing the solution through an electrolyzer, produce ammonia which may be used as a general purpose cleaner for surfaces and objects.

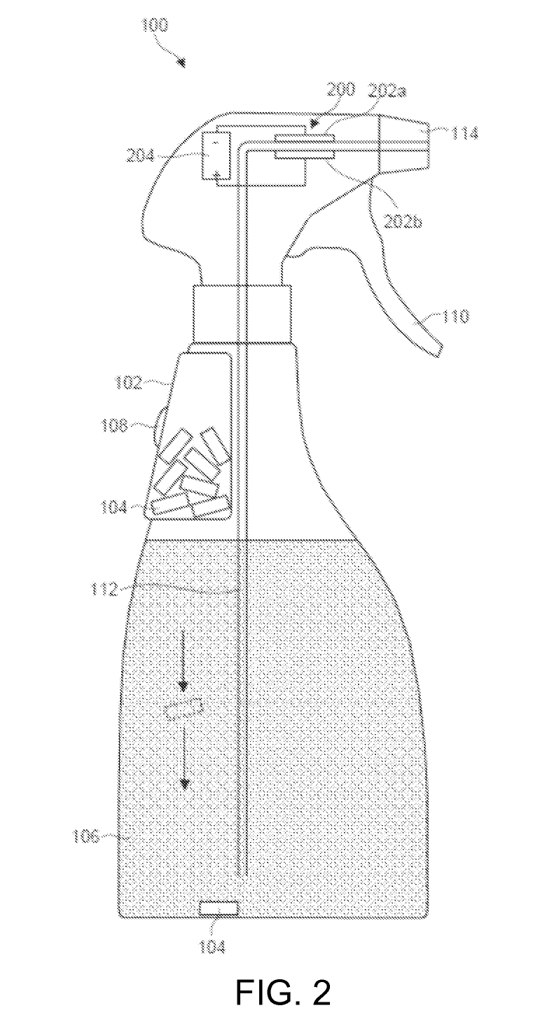

In certain embodiments, the pellets 104 or tablets 104 contain quaternary ammonium salts. The spray bottle 100 may contain normal tap water and a quaternary salt in the form of a pellet 104. The pellet dissolves in the tap water and the resulting solution is then passed through an electrolyzer (see FIG. 2) within the spray bottle 100 head. As discussed below, the electrolyzer consists of an anode and a cathode. In one embodiment, the anode may be constructed from a dimensionally stable carbon-based material and the cathode may be a composite constructed from nickel alloy metal. When the tablet 104 dissolves in the water, it forms a solution of quaternary ammonium salt, which passes through the electrolyzer. The electrolyzer dissociates the quaternary ammonium salt solution such that the halide component of the quaternary ammonium salt is converted to its corresponding halogen. The halogen dissolves in the solution producing hypohalous acid and hypohalite according the following equation:

NR₄+X+H₂O→NR₄+XO+H₂↑

Where NR₄⁺ is the quaternary ammonium ion containing a central nitrogen atom connecting four hydrocarbon functional groups denoted by R. X is a halide ion, which could be for example, one or more of Cl⁻, Br⁻, I⁻ and the like. XO⁻ is a hypohalite ion, which could be for example, one or more of ClO⁻, BrO⁻, IO⁻ and the like. In one embodiment, the quaternary ammonium salt may include one or more of benzakonium chloride, benzethonium chloride, methylbenzethonium chloride, cetalkonium chloride, cetylpyridinium chloride, cetrimonium, cetrimide, didecyldimethly ammonium chloride, dofanium chloride, tetraethylammonium bromide, tetramethylammonium bromide, tetramethylammonium chloride, tetrapropylammonium bromide, tetrabutylammonium bromide, tetrabutylammonium bisulfate, benzyltrimethylammonium chloride, benzyltriethylammonium chloride, benzyltriethylammonium bromide, domiphen bromide, and the like. It will be appreciated by those of skill in the art, that other quaternary ammonium halides may be used. The quaternary ammonium salt may also include polyquarternium polymers.

In one embodiment, the soluble material comprises at least one of a soluble chlorite, a soluble hypochlorite, a soluble halide, an ammonium salt, an alkali percarbonate, an alkali perborate salt, and combinations thereof. The soluble material may also include salts of silver, such as for example, silver carbonate, silver citrate, silver acetate, and combinations thereof. In other embodiments, the soluble material comprises hydrogen peroxide and a stabilizer. The stabilizer may include metal chelating agents and colloids including stannates, pyrophosphates and organophosphonates.

As shown in FIG. 1, the spray bottle 100 includes a trigger-like actuator 110. Squeezing the trigger 110 actuates a pump (not shown), which draws the solution 106 into a conduit 112 and expels the solution through a nozzle 114 onto a surface or object. The cleaning and/or disinfecting agent in the solution may lift dirt from the surface or object, and/or kill bacteria or other microorganisms residing on the surface or object.

Referring to FIG. 2, as previously mentioned, in selected embodiments, the solution 106 in the spray bottle 100 may be passed through an electrolyzer 200 to electrolyze selected compounds in the solution 106. As shown, the electrolyzer 200 includes a pair of electrodes 202a, 202b. A negative electrode 202a attracts positive ions and a positive electrode 202b attracts negative ions. In the illustrated embodiment, a battery 204 creates a potential difference between the electrodes 202a, 202b, resulting in the passage of electrical current between the electrodes 202a, 202b. In certain embodiments, additional circuitry (not shown) is added to the electrolyzer 200 that only applies a potential difference across the electrodes 202a, 202b when the trigger 110 is actuated and the solution 106 is passed between the electrodes 202a, 202b, thereby preserving energy in the battery 204.

If, for example, the solution 106 is a sodium chloride (NaCl) solution, the electrolyzer 200 may disassociate the NaCL to produce sodium ions and chlorine gas by drawing sodium to the negative electrode 202a and chlorine to the positive electrode 202b. A fraction of the current may also be utilized to split water and thereby generate oxygen and hydrogen. The sodium will plate the negative electrode 202a, thereby leaving chlorine, a powerful disinfectant, in the exiting stream or spray. A chlorine evolving electrode may be used as the positive electrode 202b to generate chlorine. Examples of chlorine-evolving electrodes include Dimensionally Stable Anode (DSA), which is a mixture of ruthenium oxide, iridium oxide, and titanium oxide deposited on titanium metal. Chlorine is effective to kill bacteria or other organisms residing on a surface or object. The chlorine in the exiting stream or spray may also emit a scent that reassures a user that disinfection is taking place. Sodium chloride represents just one example of a compound that may be disassociated by the electrodes 202a, 202b and is not intended to be limiting.

By introducing ions and gases into the solution 106, the electrolyzer 200 is effective to convert the solution 106 into an electrochemically “activated” liquid. For the purposes of this disclosure, an electrochemically “activated” liquid is a liquid with elevated reactivity that contains (1) reactive species, and/or (2) meta-stable (activated) ions and free radicals formed after exposure to electrochemical energy in the form of a substantial voltage potential or current under non-equilibrium conditions. The term “activated” means, for example, the electrochemical or electrophysical state or condition of having excessive inner potential energy that is attained after exposure to thermodynamically non-equilibrium conditions for a period of time. Meta-stable ions and free radicals relax in time by undergoing a gradual transition from a meta-stable state to a state of thermo-dynamic equilibrium.

In the case of electrochemically activated water, the initial liquid source used to form electrochemically activated water may include, for example, (1) regular, untreated tap water or other water that is commonly available, (2) pure water to which one or more electrolytes have been added, (3) chemically treated tap water, and (4) other aqueous solutions containing a suitable concentration of electrolytes. Examples of suitable electrolytes include chloride salt, nitrate salt, carbonate salt, or any other salt that is soluble in water (or other liquid being electrochemically activated). Chloride salts include, for example, sodium chloride (such as pure NaCl), potassium chloride, magnesium chloride, calcium chloride, and the like. The term “electrolyte” means any substance that dissociates into two or more ions when dissolved in water or any substance that will conduct an electric current when in solution.

Electrochemically activated water has enhanced cleaning power and sanitation capability compared to non-electrochemically activated water. Electrochemically activated water also differs from regular or untreated water at the molecular level and electron level. It should also be noted that adding fine gas bubbles to electrochemically activated water creates a cleaning liquid that can efficiently wet a surface. If a reactive gas is used, such as oxygen, the oxygen gas bubbles can improve the wetting properties of the liquid by reducing the surface tension of the liquid and can be reactive to further enhance the cleaning and/or sanitizing properties of the liquid. The end result is an electrochemically activated foam, froth, or reactive gas with enhanced cleaning and/or sanitizing power.

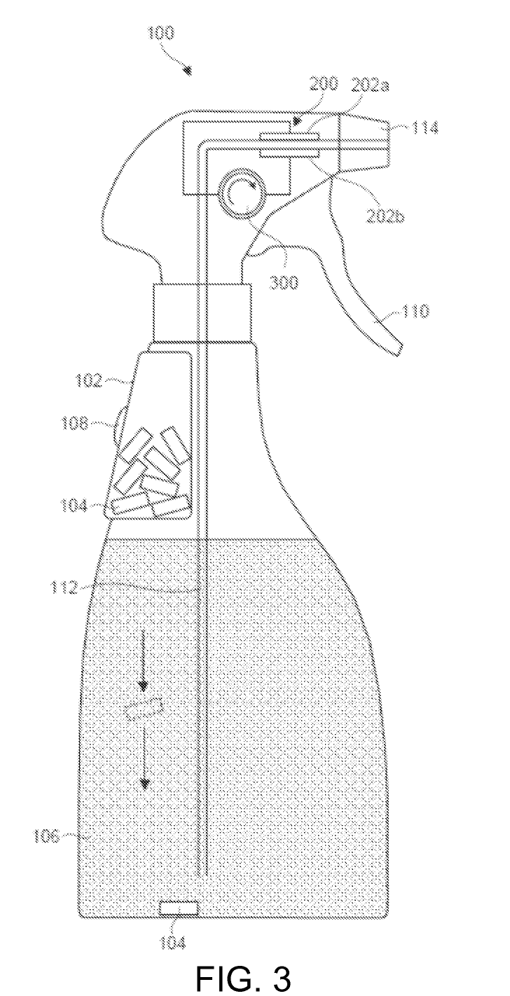

Referring to FIG. 3, in another embodiment, the electrolyzer 200 may be powered by a generator 300, such as a generator 300 actuated by the trigger 110. More particularly, actuating the trigger 110 may cause the generator 300 to spin and create electricity. In certain embodiments, a flywheel may be coupled to the generator 300 such that, when a user spins the generator 300, the flywheel will keep the generator 300 spinning for some designated period of time, such as a second or two. In other embodiments, the generator 300 may spin sufficiently without a flywheel. When a user squeezes the trigger 110, two actions may occur simultaneously: first, power will be supplied to the electrodes 202a, 202b, thereby splitting selected compounds in the solution 106; second, a pump is driven to produce a stream or spray, containing the disassociated ions, from the nozzle 114.

The spray bottle 100 of FIG. 3 is advantageous in that it does not require a battery 204 that may need to be periodically replaced or recharged. Furthermore, the generator 300 only produces power when needed—when the solution is being sprayed from the spray bottle 100. The trigger 110 converts a user's mechanical energy to the power needed to both spray and electrolyze the solution 106 simultaneously.

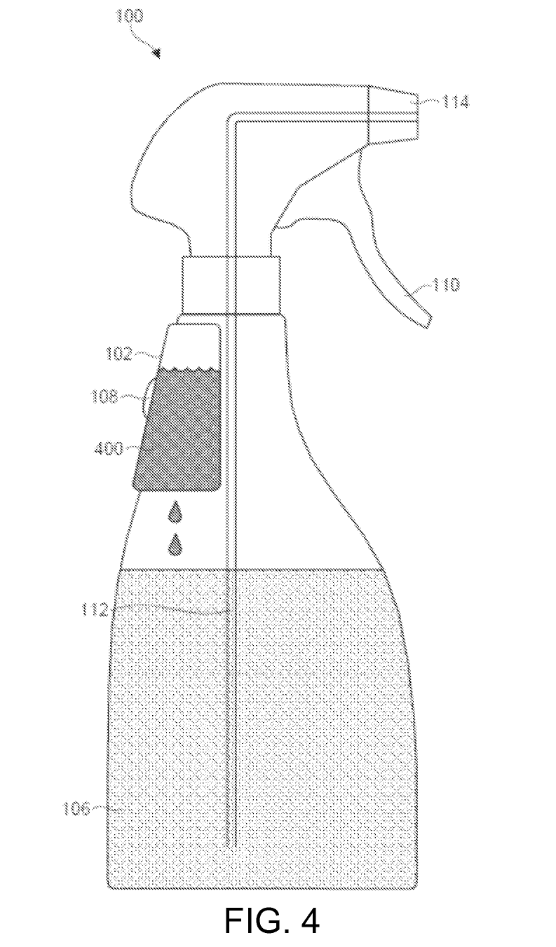

Referring to FIG. 4, in certain embodiments, instead of dispensing pellets 104 or tablets 104, the integrated dispenser 102 may be configured to dispense a liquid or gel into the spray bottle 100. The liquid or gel contains a cleaning agent and/or disinfecting agent that dissolves in or mixes with water to form a solution 106. The solution 106 may have disinfecting/cleaning properties, or have such properties after the solution 106 is passed through an electrolyzer 200 or an electrochemical cell, as previously discussed. In certain embodiments, pressing a button 108 on the integrated dispenser 102 will release one or more drops of the liquid 400 or gel 400 into the spray bottle 100.

In certain embodiments, the liquid 400 or gel 400 is a concentrated cleaning and/or disinfecting solution that becomes more dilute when it is released into a larger volume of water. The concentrated cleaning and/or disinfecting solution 400 may contain any of the chemicals discussed above with respect to FIG. 1. When the solution 106 is consumed, a user simply refills the spray bottle 100 with water and releases additional liquid 400 or gel 400 into the water, thereby producing additional cleaning/disinfecting solution 106. The integrated dispenser 102 may contain enough concentrated cleaning solution 400 for multiple refills of the spray bottle 100. Thus, like the previous example, the user will only need to have water at his or her disposal to replenish the spray bottle 100 with cleaning and/or disinfecting solution 106.

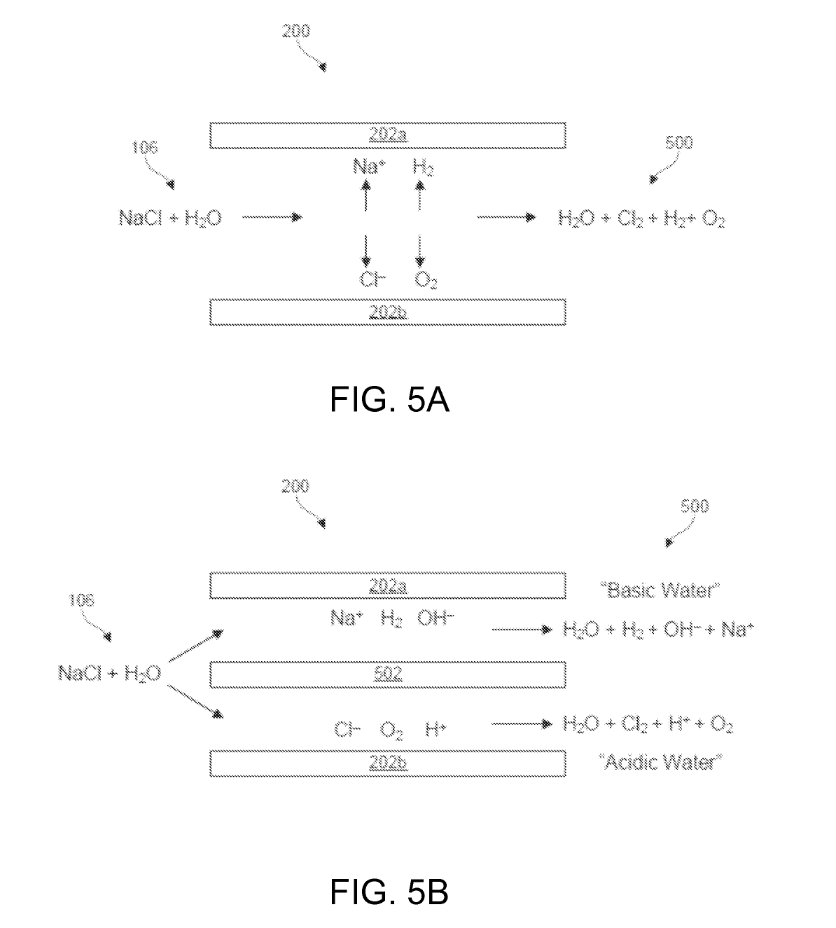

Referring to FIG. 5A, as discussed above, the cleaning and/or disinfecting solution 106 may, in certain embodiments, be passed through an electrolyzer 200 to create or enhance the cleaning and/or disinfecting properties of the solution 106. FIG. 5A shows one example of an electrolyzer 200 receiving a solution 106 of water and sodium chloride (NaCl). As shown, upon receiving the solution 106, the electrodes 202a, 202b decompose the sodium chloride compound. The negative electrode 202a attracts sodium ions (Na⁺) and the positive electrode 202b attracts chlorine ions (Cl⁻). The sodium plates the negative electrode 202a whereas the positive electrode 202b generates chlorine-based mixed oxidants. The chlorine-based mixed oxidants include predominantly chlorine gas (Cl₂), but also hypochlorite, chlorine dioxide, chlorate, perchlorate, and other oxidized chlorine-containing species, which leave the electrolyzer 200 in the exiting stream 500. In certain embodiments, the electrolyzer 200 may also decompose water in the solution 106 to generate some hydrogen, oxygen, and ozone gas in the exiting stream 500. When chlorine gas (Cl₂) combines with water in the exiting stream 500, a dilute mixture of hypochlorous acid (HOCl) and hydrochloric acid (HCl) may be generated in accordance with the following equation:

Cl₂+H₂O→HOCl+HCl

Both hypochlorous acid (HOCl) and hydrochloric acid (HCl) have antimicrobial properties and are used for cleaning and disinfecting. Any ozone generated also has disinfecting properties.

Referring to FIG. 5B, in certain embodiments, an ion-exchange membrane 502 may be present between the electrodes 202a, 202b to divide the cell 200 into anode and cathode compartments. If the ion-exchange membrane 502 is of anionic type, then it only allows anions to migrate from the negative electrode 202a to the positive electrode 202b. If the ion-exchange membrane 502 is of cationic type, then it only allows cations to migrate from the positive electrode 202b to the negative electrode 202a. Examples of cationic membranes 502 include NaSICON and Nafion® and examples of anionic membranes 502 include ACS (from Tokuyama corp., Japan) and AMI (from Membranes International). The advantage of this embodiment compared to the embodiment of FIG. 5A is that acidic water is generated in the anode compartment and basic water is generated in the cathode compartment. In the case where no membrane 502 is present the acidic and basic waters combine in the electrolyzer 200. The acidic and basic streams can be separately delivered to the area to be cleaned where they can combine. Thus water with more ions can be generated when a membrane 502 is present resulting in stronger cleaning and disinfecting action.

It should be recognized that the chemical reactions presented in FIGS. 5A and 5B are presented only by way of example and not limitation. The input stream 106 may contain different chemicals which may, in turn, produce different chemicals in the output stream 500. FIGS. 5A and 5B are simply shown to provide examples of how an electrolyzer 200 may be used to alter a solution 106 in a spray bottle 100 to enhance or change the disinfecting/cleaning properties of the solution 106.

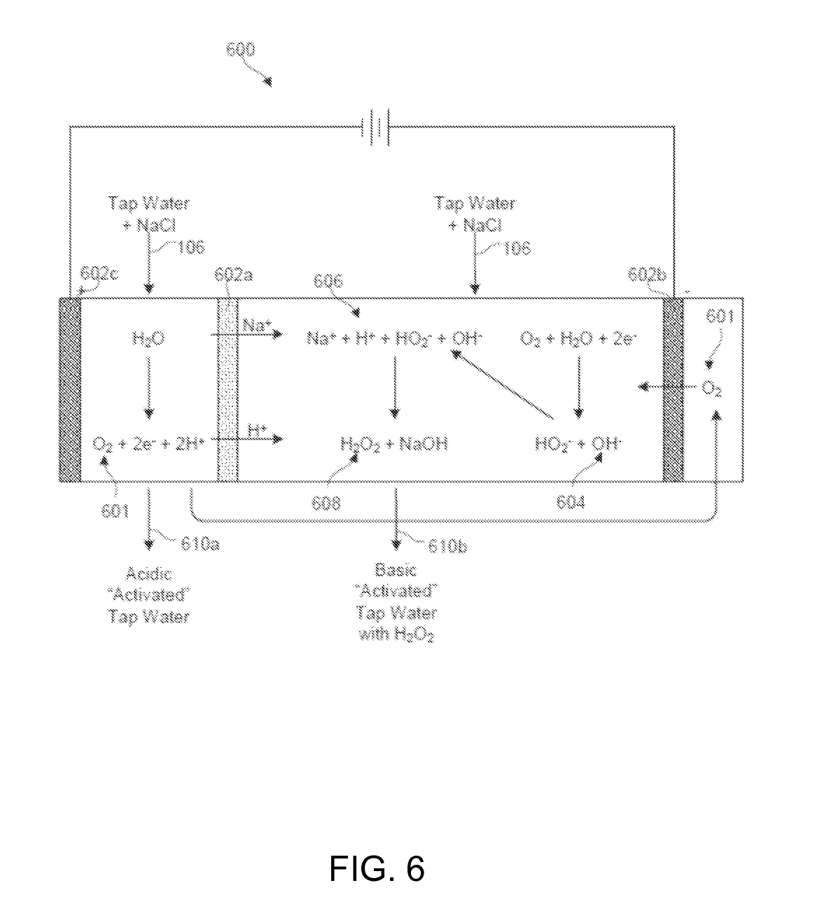

Referring to FIG. 6, one embodiment of an electrochemical cell 600 for producing “activated” water containing hydrogen peroxide is illustrated. Such an electrochemical cell 600 may be used in place of or in conjunction with the electrolyzer 200 previously described. The electrochemical cell 600 may also be considered an electrolyzer 200 for the purposes of the specification and claims.

FIG. 6 shows an electrochemical cell 600 that produces “activated” water which contains hydrogen peroxide, a well-known disinfectant. As shown, in one embodiment, the electrochemical cell 600 receives a solution 106 of tap water and sodium chloride. In certain embodiments, the tap water contains enough sodium chloride that no additional sodium chloride needs to be added. Upon receiving the tap water with sodium chloride, a water splitting reaction occurs at the anode 602c resulting in the generation of oxygen gas 601 and protons. Protons and sodium ions are transported through an ionically conductive membrane 602a. The oxygen gas 601 generated at the anode 602c is then transported to a Gas Diffusion Electrode (GDE) 602b (acting as a cathode 602b) where it reduces to form peroxide ions 604. The construction of a GDE is well known to those skilled in the art. The peroxide ions 604 then react with hydrogen ions 606 (which have been previously transported through the membrane 602a) to produce hydrogen peroxide 608 (H₂O₂). In this way, the electrochemical cell 600 produces two output streams 610a, 610b: (1) acidic “activated” tap water with chlorine-based mixed oxidants; and (2) basic “activated” tap water with hydrogen peroxide. These two output streams 610a, 610b may be independently or in combination sprayed from the spray bottle 100 to clean and disinfect a surface or object.

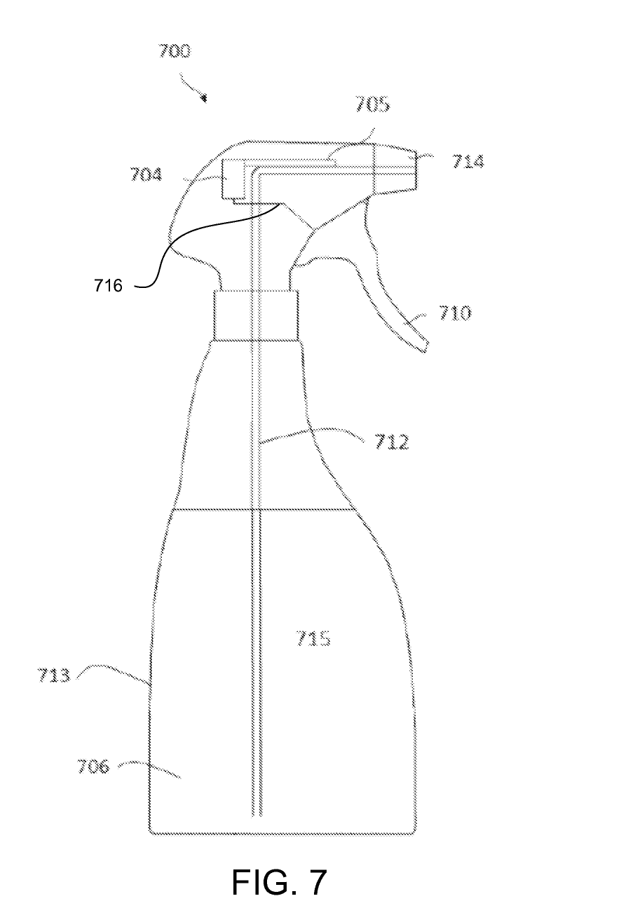

Referring now to FIG. 7, an apparatus for cleaning and/or disinfecting surfaces and objects is disclosed. The apparatus includes a spray bottle 700 that is refillable with an aqueous solution 706. The spray bottle 700 includes a nozzle 714 and a container 713. The container is configured to hold the aqueous solution 706. In one embodiment, the container 713 is one of plastic, metal, alloys and polymers, or a combination thereof. The container 713 can be made from any number of materials that do not react to the aqueous solution 706. A conduit 712 is in communication with the nozzle 714 and an interior 715 of the container 713.

The spray bottle 700 also includes an actuator 710 for pumping the aqueous solution 706 from the interior 715 of the container 713 to outside the spray bottle 700 through the nozzle 714. An ultraviolet light source 705 is in communication with the conduit 712 and is configured to at least partially radiate the aqueous solution 706 in the conduit 712. In some embodiments, the radiation creates a plurality of hydroxyl radicals in the solution, which act as sterilizing agents. A power source 704 is in communication with the ultraviolet light source 705. The spray bottle 700 includes an actuator 710 in electrical communication 716 with the power source 704, the actuator 710 configured to provide power to the ultraviolet light source 705 upon actuation of the actuator 710. In one embodiment the aqueous solution 706 includes at least one of a sodium percarbonate, a sodium perborate, and a hydrogen peroxide.

The embodiment of FIG. 7 may also include a dispenser of the type shown in FIG. 2 and the other features shown and described in FIG. 2. The dispenser may include pellets (see FIG. 2) that can mix with water in the container 713 to form the solution 706. In one embodiment, the pellets are sodium percarbonate or sodium perborate. In other embodiments, there is no dispenser and the sodium percarbonate or sodium perborate is put into the container 713 at the time of usage. In other embodiments, hydrogen peroxide is put into the container 713 of the spray bottle 700. It will be appreciated that when hydrogen peroxide, either purchased separately and put into the container 713, or generated by dissolving sodium percarbonate or perborate, passes through the ultraviolet lights source 705, hydroxyl radicals are formed. In some embodiments, the embodiment of FIG. 7 further includes an electrolyzer configured to at least partially electrolyze the aqueous solution 706 to produce a sterilizing material, as is described more fully above. In some embodiments, the electrolyzer is configured to dissociate a quaternary ammonium salt solution such that a halide component of the quaternary ammonium salt is converted to its corresponding halogen. In some embodiments, the halogen dissolves in solution producing hypohalous acid and hypohalite according the following equation:

NR₄+X+H₂O→NR₄+XO+H₂↑

where NR₄⁺ is the quaternary ammonium ion containing a central nitrogen atom connecting four hydrocarbon functional groups denoted by R, X is a halide ion, and XO⁻ is a hypohalite ion.

Some embodiments include both the ultraviolet light source 705 and the electrolyzer as described more fully in conjunction with FIGS. 2 and 3. In some embodiments, the electrolyzer and ultraviolet light source 705 are in communication with the same conduit and are placed in series. In some embodiments, the electrolyzer is placed before the ultraviolet light source 705. In some embodiments, the electrolyzer is placed after the ultraviolet light source 705. In some embodiments, the electrolyzer and ultraviolet light source 705 are placed in parallel. Some embodiments include more than one conduit 712 and the electrolyzer and the ultraviolet light source 705 are in communication with separate conduits. In some embodiments, the separate conduits recombine into a single conduit before the aqueous solution is expelled through the nozzle.

In some embodiments, the aqueous solution 706 includes an ammonium salt. In some embodiments, the ammonium salt includes a quaternary ammonium salt. In some embodiments, the quaternary ammonium salt includes a halide. In some embodiments, the quaternary ammonium salt includes one of the following: benzakonium chloride, benzethonium chloride, methylbenzethonium chloride, cetalkonium chloride, cetylpyridinium chloride, cetrimonium, cetrimide, didecyldimethly ammonium chloride, dofanium chloride, tetraethylammonium bromide, tetramethylammonium bromide, tetramethylammonium chloride, tetrapropylammonium bromide, tetrabutylammonium bromide, tetrabutylammonium bisulfate, benzyltrimethylammonium chloride, benzyltriethylammonium cloride, benzyltriethylammonium bromide, and domiphen bromide. In some embodiments, the aqueous solution 706 includes one of silver carbonate, silver citrate, and silver acetate, or combinations thereof. In some embodiments, the aqueous solution 706 includes hydrogen peroxide and a stabilizer. The features and description of each figure described herein may be combined collectively into one embodiment. Additionally, the features and description of each figure may combined with the features and description of one, two, or more figures, or in any combination (e.g., the features and description of FIG. 7 may be combined with the features and description of FIG. 3, etc.)

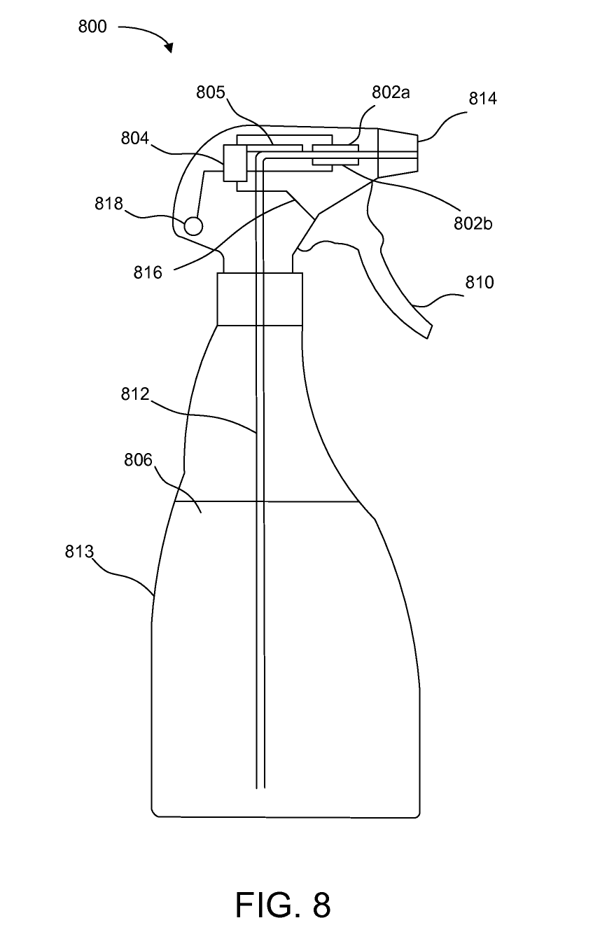

FIG. 8 depicts an embodiment of an apparatus for cleaning and/or disinfecting surfaces and objects. The embodiment includes an ultraviolet light source and an electrolyzer incorporated into a spray bottle. The apparatus includes a spray bottle 800 that is refillable with an aqueous solution 806. The spray bottle 800 includes a nozzle 814 and a container 813. The container is configured to hold the aqueous solution 806. In one embodiment, the container 813 is one of plastic, metal, alloys and polymers, or a combination thereof. The container 813 can be made from any number of materials that do not react to the aqueous solution 806. A conduit 812 is in communication with the nozzle 814 and an interior of the container 813.

The spray bottle 800 also includes an actuator 810 for pumping the aqueous solution 806 from the interior of the container 813 to outside the spray bottle 800 through the nozzle 814. An ultraviolet light source 805 is in communication with the conduit 812 and is configured to at least partially radiate the aqueous solution 806 in the conduit 812. In some embodiments, the radiation creates a plurality of hydroxyl radicals in the solution, which act as sterilizing agents. A power source 804 is in communication with the ultraviolet light source 805. The spray bottle 800 includes an actuator 810 in electrical communication 816 with the power source 804, the actuator 810 configured to provide power to the ultraviolet light source 805 upon actuation of the actuator 810. In one embodiment the aqueous solution 806 includes at least one of a sodium percarbonate, a sodium perborate, and a hydrogen peroxide. In addition, the spray bottle includes an electrolyzer, including a pair of electrodes 802a and 802b, in communication with the conduit 812. The illustrated embodiment depicts the ultraviolet light source 805 and the electrolyzer in series, with the flowing aqueous solution 805 within the conduit 812 in communication with the ultraviolet light source 805 prior to the electrolyzer. In some embodiments, the electrolyzer is located prior to the ultraviolet light source 805. The illustrated embodiment includes a switch 818. In some embodiments, the switch 818 may allow a user to activate and/or deactivate either the electrolyzer or the ultraviolet light source 805 or both. For example, activating the switch may cause the power source to generate power to only one of the electrolyzer or the ultraviolet light source 805.

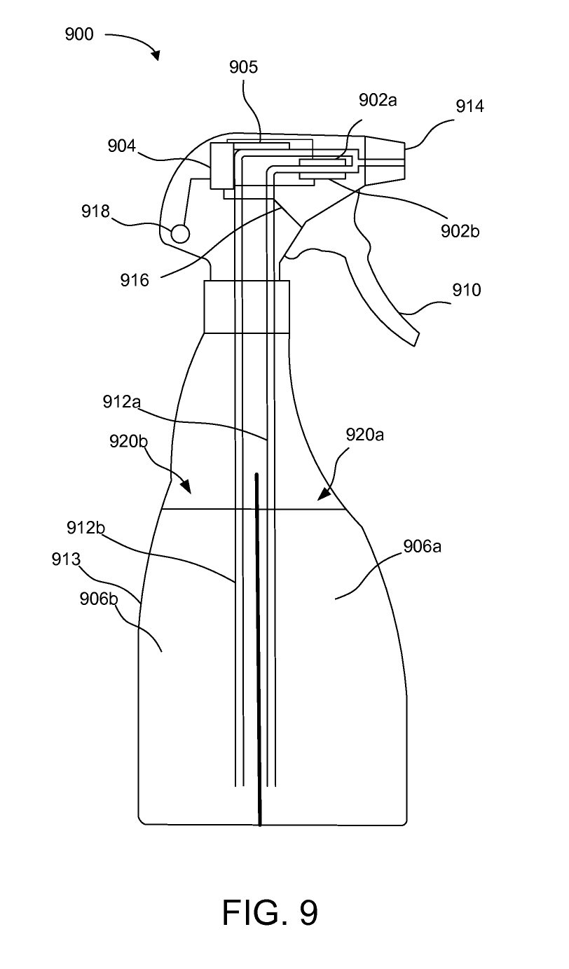

FIG. 9 depicts an embodiment of an apparatus for cleaning and/or disinfecting surfaces and objects. The embodiment includes an ultraviolet light source and an electrolyzer incorporated into a spray bottle. The apparatus includes a spray bottle 900 that is refillable with two aqueous solutions 906a and 906b located in separate compartments 920a and 920b. The spray bottle 900 includes a nozzle 914 and a container 913 including the separate compartments 920a and 920b. The container is configured to hold the aqueous solution 906a in compartment 920a and the aqueous solution 906b in compartment 920b. In one embodiment, the container 913 is one of plastic, metal, alloys and polymers, or a combination thereof. The container 913 can be made from any number of materials that do not react to the aqueous solutions 906a and 906b. The spray bottle may include two conduits 912a and 912b which are in communication with the nozzle 914 and an interior of the compartments 920a and 920b. In the illustrated embodiment, the conduits 912a and 912b recombine into a single conduit before the nozzle 914.

The spray bottle 900 also includes an actuator 910 for pumping the aqueous solution 906a, the aqueous solution 906b, or both from their respective compartments 920a and 920b to outside the spray bottle 900 through the nozzle 914. An ultraviolet light source 905 is in communication with the conduit 912b and is configured to at least partially radiate the aqueous solution 906b in the conduit 912b. In some embodiments, the radiation creates a plurality of hydroxyl radicals in the solution, which act as sterilizing agents. A power source 904 is in communication with the ultraviolet light source 905. The spray bottle 900 includes an actuator 910 in electrical communication 916 with the power source 904, the actuator 910 configured to provide power to the ultraviolet light source 905 upon actuation of the actuator 910. In one embodiment the aqueous solution 906a includes at least one of a sodium percarbonate, a sodium perborate, and a hydrogen peroxide. In some embodiments, the spray bottle 900 further includes an electrolyzer, including a pair of electrodes 902a and 902b, in communication with the conduit 912a. The illustrated embodiment depicts the ultraviolet light source 905 and the electrolyzer in parallel. In some embodiments, the ultraviolet light source 905 and the electrolyzer are in parallel with a single aqueous solution entering both conduits. The illustrated embodiment includes a switch 918. In some embodiments, the switch 918 may allow a user to activate and/or deactivate either the electrolyzer or the ultraviolet light source 805 or both, as well as activate and/or deactivate pumping from one or both compartments 920a and 920b. For example, activating the switch may cause the power source to generate power to only one of the electrolyzer or the ultraviolet light source 905 and only pump aqueous solution from the corresponding compartment.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. An apparatus for cleaning and/or disinfecting surfaces and objects, the apparatus comprising:
a spray bottle that is refillable with an aqueous solution, the spray bottle comprising a nozzle and a container;
a conduit in communication with the nozzle and an interior of the container;
an actuator for pumping the aqueous solution from the interior of the container to outside the spray bottle through the nozzle;
an ultraviolet light source in communication with the conduit configured to at least partially radiate the aqueous solution in the conduit;
a power source in communication with the ultraviolet light source; and
an actuator in electrical communication with the power source, the actuator configured to provide power to the ultraviolet light source upon actuation of the actuator.

2. The apparatus of claim 1, wherein the aqueous solution comprises at least one of a sodium percarbonate, a sodium perborate, and a hydrogen peroxide.

3. The apparatus of claim 1, further comprising an electrolyzer, wherein the spray bottle is configured to pass the solution through the electrolyzer to at least partially electrolyze the soluble material to produce a sterilizing material.

4. The apparatus of claim 3, wherein the electrolyzer is configured to dissociate a quaternary ammonium salt solution such that a halide component of the quaternary ammonium salt is converted to its corresponding halogen.

5. The apparatus of claim 4, wherein the halogen dissolves in solution producing hypohalous acid and hypohalite according the following equation:
NR₄+X+H₂O→NR₄+XO+H₂↑
where NR₄⁺ is the quaternary ammonium ion containing a central nitrogen atom connecting four hydrocarbon functional groups denoted by R, X is a halide ion, and XO⁻ is a hypohalite ion.

6. The apparatus of claim 1, wherein the aqueous solution comprises an ammonium salt, wherein the ammonium salt comprises a quaternary ammonium salt.

7. The apparatus of claim 6, wherein the quaternary ammonium salt comprises a halide.

8. The apparatus of claim 6, wherein the quaternary ammonium salt comprises one of benzakonium chloride, benzethonium chloride, methylbenzethonium chloride, cetalkonium chloride, cetylpyridinium chloride, cetrimonium, cetrimide, didecyldimethly ammonium chloride, dofanium chloride, tetraethylammonium bromide, tetramethylammonium bromide, tetramethylammonium chloride, tetrapropylammonium bromide, tetrabutylammonium bromide, tetrabutylammonium bisulfate, benzyltrimethylammonium chloride, benzyltriethylammonium chloride, benzyltriethylammonium bromide, and domiphen bromide.

9. The apparatus of claim 1, wherein the aqueous solution comprises at least one of silver carbonate, silver citrate, and silver acetate.

10. The apparatus of claim 1, wherein the aqueous solution comprises hydrogen peroxide and a stabilizer.

11. An apparatus for cleaning and/or disinfecting surfaces and objects, the apparatus comprising:
a spray bottle that is refillable with water, wherein the spray bottle comprises an ultra violet light source; and
a dispenser integrated into the spray bottle to dispense a soluble material into the water to produce a solution, the soluble material comprising one of a cleaning agent and a disinfecting agent, the soluble material provided in a quantity sufficient to last several refills of the spray bottle, wherein the ultra violet light source is configured to at least partially radiate the solution to create a plurality of hydroxyl radicals in the solution.

12. The apparatus of claim 11, wherein the soluble material comprises silver carbonate, silver citrate, silver acetate, and combinations thereof.

13. The apparatus of claim 11, wherein the soluble material comprises at least one of a soluble chlorite, a soluble hypochlorite, a soluble halide, an ammonium salt, an alkali percarbonate, an alkali perborate salt, and combinations thereof.

14. The apparatus of claim 11, wherein the soluble material comprises an ammonium salt, wherein the ammonium salt comprises a quaternary ammonium salt.

15. The apparatus of claim 14, wherein the quaternary ammonium salt comprises a halide.

16. The apparatus of claim 14, wherein the quaternary ammonium salt comprises one of benzakonium chloride, benzethonium chloride, methylbenzethonium chloride, cetalkonium chloride, cetylpyridinium chloride, cetrimonium, cetrimide, didecyldimethly ammonium chloride, dofanium chloride, tetraethylammonium bromide, tetramethylammonium bromide, tetramethylammonium chloride, tetrapropylammonium bromide, tetrabutylammonium bromide, tetrabutylammonium bisulfate, benzyltrimethylammonium chloride, benzyltriethylammonium chloride, benzyltriethylammonium bromide, and domiphen bromide.

17. The apparatus of claim 11, wherein the soluble material comprises hydrogen peroxide and a stabilizer.

18. The apparatus of claim 11, further comprising an electrolyzer, wherein the spray bottle is configured to pass the solution through the electrolyzer to at least partially electrolyze the soluble material to produce a sterilizing material.

19. The apparatus of claim 18, wherein the electrolyzer is configured to dissociate a quaternary ammonium salt solution such that a halide component of the quaternary ammonium salt is converted to its corresponding halogen.

20. The apparatus of claim 19, wherein the halogen dissolves in solution producing hypohalous acid and hypohalite according the following equation:
NR₄+X+H₂O→NR₄+XO+H₂↑
where NR₄⁺ is the quaternary ammonium ion containing a central nitrogen atom connecting four hydrocarbon functional groups denoted by R, X is a halide ion, and XO⁻ is a hypohalite ion.
